(19) United States
(12) Patent Application Publication
(10) Pub. No.: US 2023/0360731 A1
(43) Pub. Date: Nov. 9, 2023

(54) SYSTEM AND METHOD FOR INTERACTIVE PATHOGEN DETECTION

(71) Applicant: The Board of Regents for the Oklahoma Agricultural and Mechanical Colleges, Stillwater, OK (US)

(72) Inventors: Kitty Frances Cardwell, Stillwater, OK (US); Andres Sebastian Espindola Camacho, Stillwater, OK (US); Tyler Dang, Rowland Heights, CA (US); Joshua Daniel Habiger, Stillwater, OK (US); Huizi Wang, Stillwater, OK (US)

(21) Appl. No.: 18/299,560

(22) Filed: Apr. 12, 2023

Related U.S. Application Data
(63) Continuation of application No. PCT/US21/55156, filed on Oct. 15, 2021.
(60) Provisional application No. 63/092,815, filed on Oct. 16, 2020.

(51) Int. Cl. G16B 30/10; G16B 30/20

(52) U.S. Cl. CPC G16B 30/10; G16B 30/20

(57) ABSTRACT

Systems and methods for interactive pathogen detection are described including receiving at least one target genome file and at least one near-neighbor genome file and analyzing the target genome file and the near-neighbor genome file to generate a plurality of raw e-probes unique to a target pathogen. Each raw e-probe includes a unique nucleic acid signature sequence selected from along a length of the pathogen genome of the target pathogen. The plurality of raw e-probes are curated to provide a curated e-probe set. The curated e-probe set can be in silico validated and/or in vitro validated. The resulting e-probe set can be used to determine presence of the target pathogen in a sample metagenome in an e-probe diagnostic system.

### REFERENCE TO RELATED APPLICATIONS

This application is a non-provisional application claiming benefit to PCT/US21/55156, filed on Oct. 15, 2021, which claims priority to U.S. Provisional Application No. 63/092,815, filed on Oct. 16, 2020, the disclosure of which is hereby incorporated by reference in its entirety.

### STATEMENT OF GOVERNMENT INTEREST

Not applicable.

### REFERENCE TO SEQUENCE LISTING SUBMITTED ELECTRONICALLY

The instant application contains, as a separate part of the present disclosure, a Sequence Listing which has been submitted via Patent Center in computer readable form as an XML file. The Sequence Listing, created Jul. 20, 2023 is named “57910198_Replacement_Sequence_Listing.xml” and is 6,152 bytes in size. The entire contents of the Sequence Listing are hereby incorporated herein by reference.

### BACKGROUND ART

Rapid and accurate pathogen detection in plants and animals aids in food security and public health. It is estimated that exotic animal and plant diseases can cost agricultural industries in the United States billions of dollars each year. Further, the lack of high throughput pathogen detection techniques and systems leaves vulnerable ports and borders open to threat of pathogen dissemination. Even local trade has the potential to disseminate pathogens. Current proactive measures to avoid the spread of disease within the art involve extensive testing limited by the cost and throughput capacity of particular technology.

Sequence-based detection technology is being explored by multiple plant quarantine agencies around the world. Until recently, nucleic acid sequencing for diagnostics has been constrained by cost, data volume, and limited bioinformatic tools for analysis. Next Generation Sequencing (NGS) data suffers from a large amount of computational time and power needed to identify a pathogen sequence from an obtained NGS dataset.

High throughput sequencing (HTS) is a powerful technology that combines molecular biology and computer sciences. HTS has been used in various applications and not just as a research tool for gene expression studies or the discovery of new unknown pathogens. The technology has gained traction and shows potential as a routine plant diagnostic method for the detection and identification of pathogens. The proper implementation of HTS diagnostic can streamline the laboratory diagnostics and progressively phase out the more than twenty individual laboratory tests (polymerase chain reaction (PCR), quantitative PCR (qPCR), enzyme-linked immunoassay (ELISA), and the like) currently required for the detection of all known citrus graft-transmissible citrus pathogens, for example. HTS can generate data with enough resolution to discern between different isolates of the same pathogen. In addition, the HTS technology may allow for the reduction of plant indicators used for biological indexing that has the capability to free valuable greenhouse space. With the constant declining cost of HTS, it has made the technology more accessible for laboratories to implement.

One difficulty with implementation of HTS diagnostics is the data analysis, as data analysis is time consuming, laborious, and requires dedicated personnel with high-level knowledge in bioinformatics and computer programming as well as access to expensive high performance computing. Cut off for diagnosis calls using a traditional bioinformatic workflow (aligning, assembling and BLASTn reads) can vary between lab to lab and in some cases be arbitrary. The current online Virfind platform provides a user-friendly bioinformatic pipeline that can be used for pathogen detection; however, the analysis can be over complicated because of excess information that needs to be sorted by the user and the inclusion of unrelated or unknown pathogens which are not necessarily regulated.

To overcome challenges with HTS data analysis, the MiFi® platform originally developed by Oklahoma State University Institute of Biosecurity and Microbial Forensic provides a user-friendly online HTS data analysis tool for diagnostic applications. The MiFi® platform is a bioinformatic tool that utilizes short curated electronic probes (e-probes) designed from pathogen specific sequences. The e-probes are used to detect and/or identify a single or multiple pathogens of interest from raw HTS datasets and ignore irrelevant sequences such as the host or other microbes present in the sample.

The ability to simultaneously screen for multiple or all possible pathogens within a sample may enable a more timely response, as well as, aid in mitigation and management of potential plant, animal and human disease introductions and outbreaks.

### BRIEF DESCRIPTION OF THE DRAWINGS

FIG. 1 illustrates a block diagram of an exemplary interactive pathogen detection system in accordance with the present disclosure.

FIG. 2 illustrates another block diagram of the exemplary interactive pathogen system illustrated in FIG. 1.

FIG. 3 illustrates a flow diagram of an exemplary method for design of e-probes via an e-probe design system of the interactive pathogen detection system in accordance with the present disclosure.

FIG. 4A is a table including pathogens of grapevine, associated National Center for Biotechnology Information (NCBI) taxon identifications (ID) for the pathogens of grapevine, and total number of raw e-probes designed by the e-probe design system for the pathogens of grapevine in accordance with the present disclosure.

FIG. 4B is a table including pathogens of citrus, total number of raw e-probes designed by the e-probe design system for the pathogens of citrus, and theoretical limit of detection (LOD) associated with the e-probes in accordance with the present disclosure.

FIG. 5A is a graphical linear regression showing relationship of e-probe hits with simulated relative prevalence of a virus in a metagenome, comparing fifteen raw e-probes before curation and five curated e-probes after curation of Grapevine Leafroll-associated Virus 3 (GLRaV-3).

FIG. 5B is a graphical linear regression showing relationship of e-probe hits with simulated relative prevalence of a virus in a metagenome between e-probes of Dichoraviruses.

FIG. 6A is a boxplot graph depicting pathogen titer response with fifteen in silico e-probes for GLRaV-3.

FIG. 6B is a boxplot graph depicting pathogen titer response with thirteen e-probe sets for Dichoraviruses.

FIG. 7 is a flow chart of an exemplary method for determining and providing internal control e-probes for validation in accordance with the present disclosure.

FIG. 8 is a flow chart of an exemplary method for detecting one or more target pathogens in the sample metagenome using a plurality of e-probes in accordance with the present disclosure.

FIGS. 9-18 illustrate exemplary screenshots of an interactive pathogen detection system.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “pathogen” as used herein includes to any bacterium, virus and/or other microorganism capable of causing disease. The term “host” as used herein includes any organism that is infected with, fed upon by, and/or harboring a pathogenic organism including a plant supporting an epiphyte. The term “microbiome” as used herein includes the community of micro-organisms with a particular habitat.

The term “treatment” refers to both therapeutic treatment and prophylactic or preventative measures. Those in need of treatment include, but are not limited to, entities already having a particular condition/disease/infection as well as entities at risk of acquiring a particular condition/disease/infection (e.g., those needing prophylactic/preventative measures). The term “treating” refers to administering an agent/element/method for therapeutic and/or prophylactic/preventative purposes.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), a field programmable gate array (FPGA), a combination of hardware and software, and/or the like. The term “processor” as used herein means a single processor or multiple processors working independently or together to collectively perform a task.

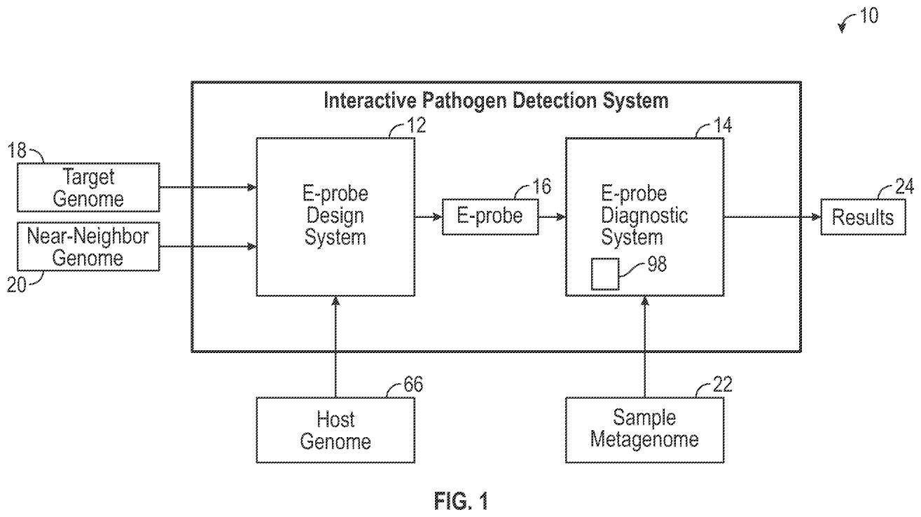

Turning now to the drawings and in particular to FIG. 1, certain non-limiting embodiments thereof include an interactive pathogen detection system 10 in accordance with the present disclosure. Generally, the interactive pathogen detection system 10 is configured to provide identification and/or characterization of one or more pathogens in a given sample (e.g., plant tissue, leaf, stem, seed, and root). In some embodiments, the interactive pathogen detection system 10 may provide identification and simultaneous characterization of the one or more pathogens in a single sample. Pathogens may include RNA virus, DNA virus, bacteria, fungi, oomycete, and/or the like. Pathogens may be plant, animal or human pathogens. In some embodiments, the interactive pathogen detection system 10 provides a crowd sourced created database configured to detect any type of pathogen or microbe within a sample.

Generally, the interactive pathogen detection system 10 includes an e-probe design system 12 and an e-probe diagnostic system 14. The e-probe design system 12 is configured to build, curate, and/or validate electronic probes (e-probes) for each pathogen of interest 16 or e-probe sets for use in the interactive pathogen detection system 10. E-probes 16 are a set of unique nucleic acid signature sequences, from 20 to 100 nucleotides long (depending on the size of the organism) selected from along the length of a pathogen genome. In particular, e-probes 16 may be designed to be very specific to closely related strains of pathogens, and still have an adequate level of sensitivity to detect a particular strain. Further, via the use of e-probes 16 in accordance with the present disclosure, a user is able to simultaneously test for different strains of pathogens within a single sample.

Generally, the e-probe design system 12 receives one or more target genomes 18 and near-neighbor genomes 20. The one or more target genomes 18 are the collection of sequences for consideration of detection (i.e., inclusivity panel) for a particular pathogen, for example. The near-neighbor genome(s) are collection of sequences for group(s) or organism(s) for exclusion of detection (i.e., exclusivity panel) for the particular pathogen (i.e., target pathogen). The e-probe design system is configured to identify unique sequences (e.g., DNA sequences, RNA sequences) present within the target genome 18 by analyzing the target genome 18 and eliminating any and all sequence matches to one or more near-neighbor genomes 20 and provide e-probes 16 based on the determined sequences. The e-probe design system 12 may be configured to assess sensitivity, specificity and/or limit of detection (LOD) of e-probes or e-probe sets for a particular microbe.

The e-probe diagnostic system 14 is configured to determine the presence or absence of one or more pathogens and/or one or more microbes in a sample metagenome 22 using e-probes 16. Generally, each e-probe 16 provided by the e-probe design system 12 may be used in the e-probe diagnostic system 14 to detect presence or absence of one or more pathogens in one or more sample metagenomes 22. To that end, the e-probe diagnostic system 14 generally provides a user with e-probe pathogen-specific options that are selected by the user to query the one or more sample metagenomes 22. The e-probe diagnostic system 14 delivers an output result 24 representative of presence of the e-probe sequences within the one or more sample metagenomes 22. The output result 24 may include a determination of positive or negative detection of one or more pathogens within the sample metagenome 22. In some embodiments, one or more reports may be provided to a user detailing the output result 24.

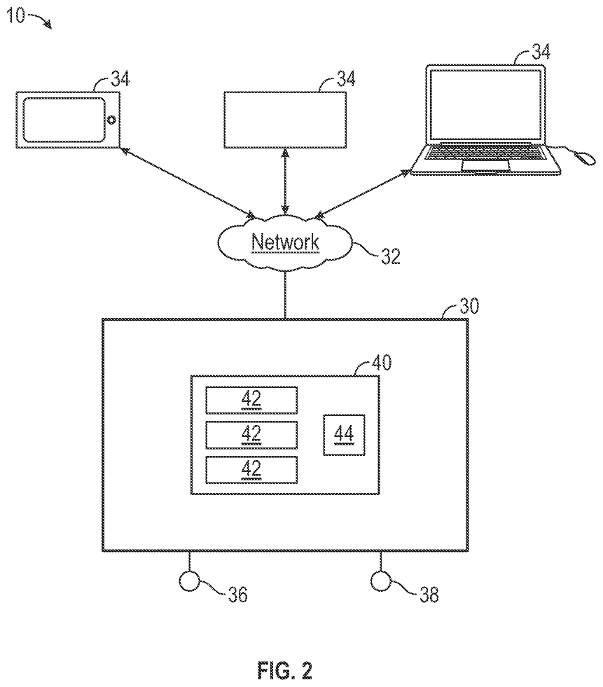

Referring to FIGS. 1 and 2, the interactive pathogen detection system 10 may be a system or systems that are able to embody and/or execute the logic of the processes described herein. Logic embodied in the form of software instructions and/or firmware may be executed on any appropriate hardware. For example, logic embodied in the form of software instructions or firmware may be executed on a dedicated system or systems, or on a personal computer system, or on a distributed processing computer system, and/or the like. In some embodiments, logic may be implemented in a stand-alone environment operating on a single computer system and/or logic may be implemented in a networked environment, such as a distributed system using multiple computers and/or processors networked together.

In some embodiments, the interactive pathogen detection system 10 may include one or more processors 30. The one or more processors 30 may work to execute processor executable code. The one or more processors 30 may be implemented as a single or plurality of processors working together, or independently, to execute the logic as described herein. Exemplary embodiments of the one or more processors 30 may include, but are not limited to, a digital signal processor (DSP), a central processing unit (CPU), a field programmable gate array (FPGA), a microprocessor, a multi-core processor, and/or combinations thereof, for example. In some embodiments, the one or more processors 30 may be incorporated into a smart device. The one or more processors 30 may be capable of communicating via a network 32 or a separate network (e.g., analog, digital, optical, and/or the like). It is to be understood, that in certain embodiments, using more than one processor, the processors 30 may be located remotely from one another, in the same location, or comprising a unitary multi-core processor. In some embodiments, the one or more processors 30 may be partially or completely network-based or cloud-based, and may or may not be located in a single physical location. The one or more processors 30 may be capable of reading and/or executing processor executable code and/or capable of creating, manipulating, retrieving, altering, and/or storing data structure into one or more memories.

In some embodiments, the one or more processors 30 may transmit and/or receive data via the network 32 to and/or from one or more external systems 34 (e.g., one or more external computer systems, one or more machine learning applications, artificial intelligence, cloud based system). For example, the one or more processors 30 may allow external systems 34 (e.g., researchers, regulators, physicians and/or medical personnel) access via the network 32 to provide and/or receive data from the one or more processors 30 (e.g., providing target genomes and/or near neighbor genomes, providing e-probe selection, providing sample metagenome, receiving positive or negative detection data). Access methods include, but are not limited to, cloud access and direct download from the one or more processors 30 via the network 32. In some embodiments, the one or more processors 30 may be provided on a cloud cluster (i.e., a group of nodes hosted on virtual machines and connected within a virtual private cloud). Additionally, processors 30 may provide data to a user by methods that include, but are not limited to, messages sent through the one or more processors 30 and/or external systems 34, SMS, email, and telephone, to provide data such as positive or negative detection data, for example. It is to be understood that in some exemplary embodiments, the one or more processors 30 and the one or more external systems 34 may be implemented as a single device.

The one or more external systems 34 may be configured to provide information and/or data in a form perceivable to a user and/or processors 30. For example, the one or more external systems 34 may include, but are not limited to, implementations as a laptop computer, a computer monitor, a screen, a touchscreen, a speaker, a website, a smart phone, a PDA, a cell phone, an optical head-mounted display, combinations thereof, and/or the like.

The one or more external systems 34 may communicate with the one or more processors 30 via the network 32. As used herein, the terms “network-based”, “cloud-based”, and any variations thereof, may include the provision of configurable computational resources on demand via interfacing with a computer and/or computer network, with software and/or data at least partially located on a computer and/or computer network, by pooling processing power of two or more networked processors.

In some embodiments, the network 32 may be the Internet and/or other network. For example, if the network 32 is the Internet, a primary user interface of the e-probe design software and/or the e-probe diagnostic software may be delivered through a series of web pages. It should be noted that the primary user interface of the e-probe design software and/or the e-probe diagnostic software may be via any type of interface, such as, for example, a Windows-based application.

The network 32 may be almost any type of network. For example, the network 32 may interface via optical and/or electronic interfaces, and/or may use a plurality of network topographies and/or protocols including, but not limited to, Ethernet, TCP/IP, circuit switched paths, combinations thereof, and the like. For example, in some embodiments, the network 32 may be implemented as the World Wide Web (or Internet), a local area network (LAN), a wide area network (WAN), a metropolitan network, a wireless network, a cellular network, a Global System of Mobile Communications (GSM) network, a code division multiple access (CDMA) network, a 4G network, a 5G network, a satellite network, a radio network, an optical network, an Ethernet network, combinations thereof, and/or the like. Additionally, the network 32 may use a variety of network protocols to permit bi-directional interface and/or communication of data and/or information. It is conceivable that in the near future, embodiments of the present disclosure may use more advanced networking topologies.

In some embodiments, the one or more processors 30 may include one or more input devices 36 and one or more output devices 38. The one or more input devices 36 may be capable of receiving information from a user, processors, and/or environment, and transmit such information to the processor 30 and/or the network 32. The one or more input devices 36 may include, but are not limited to, implementation as a keyboard, touchscreen, mouse, trackball, microphone, fingerprint reader, infrared port, slide-out keyboard, flip-out keyboard, cell phone, PDA, video game controller, remote control, network interface, speech recognition, gesture recognition, combinations thereof, and/or the like.

The one or more output devices 38 may be capable of outputting information in a form perceivable by a user, the external system 34, and/or processor(s). For example, the one or more output devices 38 may include, but are not limited to, implementations as a computer monitor, a screen, a touchscreen, a speaker, a website, a television set, a smart phone, a PDA, a cell phone, a fax machine, a printer, a laptop computer, an optical head-mounted display (OHMD), combinations thereof, and/or the like. It is to be understood that in some exemplary embodiments, the one or more input devices 36 and the one or more output devices 38 may be implemented as a single device, such as, for example, a touchscreen or a tablet.

The one or more processors 30 may be capable of reading and/or executing processor executable code and/or capable of creating, manipulating, retrieving, altering and/or storing data structures into one or more memories 40. The one or more processors 30 may include one or more non-transient memory comprising processor executable code and/or software application. In some embodiments, the one or more memories 40 may be located in the same physical location as the processor 30. Alternatively, one or more memories 40 may be located in a different physical location as the processor 30 and communicate with the processor 30 via a network, such as the network 32. Additionally, one or more memories 40 may be implemented as a “cloud memory” (i.e., one or more memories may be partially or completely based on or accessed using a network, such as network 32).

The one or more memories 40 may store processor executable code and/or information comprising one or more databases 42 and program logic 44 (i.e., computer executable logic). In some embodiments, the processor executable code may be stored as a data structure, such as a database and/or data table, for example. In some embodiments, one or more database 42 may store hypotheses and/or models related to the design of e-probes 16 and/or the detection of target pathogen(s) by the e-probe(s) obtained via the processes described herein. In use, the processor 30 may execute the program logic 44 controlling the reading, manipulation and/or storing of data as detailed in the processes described herein.

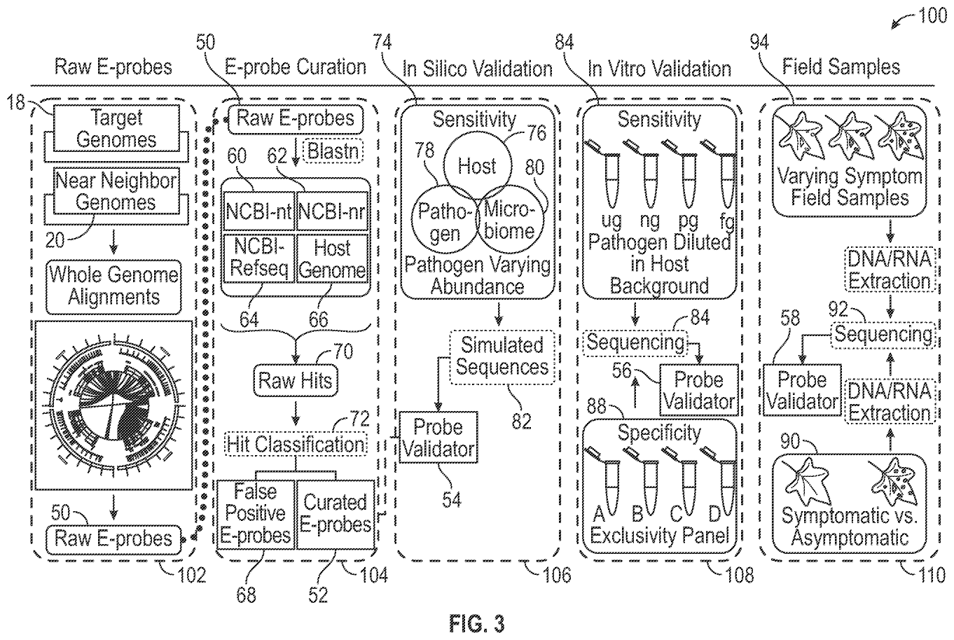

FIG. 3 illustrates a flow chart 100 of an exemplary process used by the e-probe design system 12 of FIG. 1. Generally, the e-probe design system 12 is configured to use the target genome 18 to develop, curate and validate e-probes 16 providing e-probes 16 capable of being used in the e-probe diagnostic system 14. In a step 102, the e-probe design system 12 receives one or more target genomes 18 and near-neighbor genomes 20 of a target pathogen and determines at least one set of raw e-probes 50 using the target genomes 18 and near-neighbor genomes 20. In a step 104, the e-probe design system 12 provides curated e-probe sets 52 from the set of raw e-probes 50 by eliminating one or more raw e-probe sequences 50 having distinct similarities with other pathogens and/or hosts not specific to the target pathogen. In a step 106, the e-probe design system 12 may provide in silico validated e-probes 54 from the curated e-probes 52 via in silico validation. In a step 108, the e-probe design system 12 may provide in vitro (or in vivo) validated e-probes 56 from the curated e-probes 52 and/or the in silico validated e-probes 54 via in vitro (or in vivo) validation. In some embodiments, the in silico validated e-probes 54 and/or the in vitro validated e-probes 56 may be further field validated to provide field validated e-probes 58 in a step 110. Depending on design considerations, the in silico validated e-probes 54, in vitro validated e-probes 56 and/or field validated e-probes 58 may be provided as e-probes 16 for use in the e-probe diagnostic system 14 as shown in FIG. 1.

Referring to FIGS. 2-4, in the step 102, the e-probe design system 12 determines at least one set of raw e-probes 50 using one or more target genomes 18 and one or more near-neighbor genomes 20. The target genomes 18 and the one or more near-neighbor genomes 20 may be provided by one or more users of the external systems 34 or the one or more input devices 36 of the processor 30. In some embodiments, one or more target genomes 18 for each target pathogen may be retrieved via one or more external systems 34. In some embodiments, the one or more external systems 34 may be one or more public databases including, but not limited to, the National Center for Biotechnology Information (NCBI), the European Bioinformatics Institute (EMBL), and/or any public or private genetic and/or genomic database. In some embodiments, one or more developers may generate (e.g., in situ) the one or more target genome 18 and provide the data via the one or more external systems 34. In some embodiments, the target genomes 18 and/or near-neighbor genomes 20 may be provided in a compressed file to the processor 30 to reduce upload time. In some embodiments, the target genomes 18 and/or near-neighbor genomes 20 may each be provided in a ‘fasta’ format to the processor 30. In some embodiments, the target genome 18 may be provided in a first fasta file and the one or more near-neighbor genome 20 may be provided in a second fasta file.

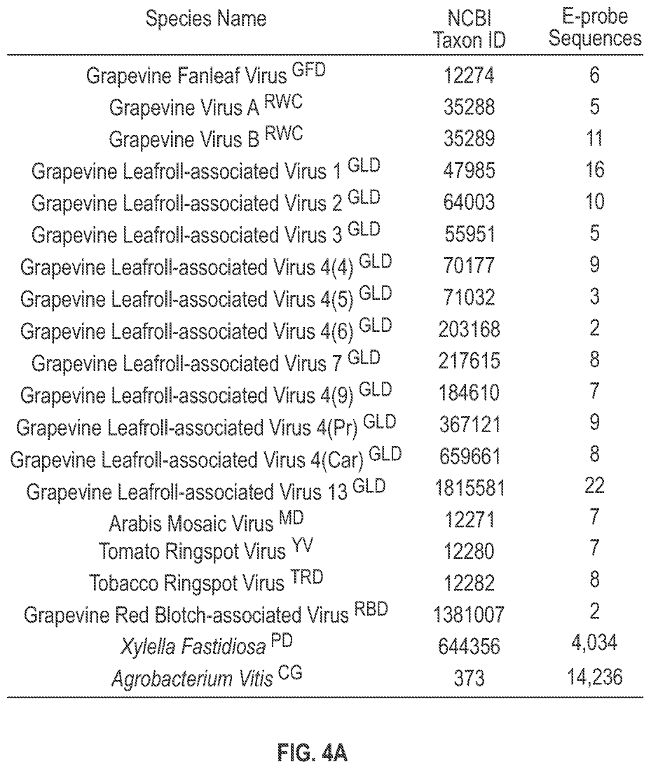

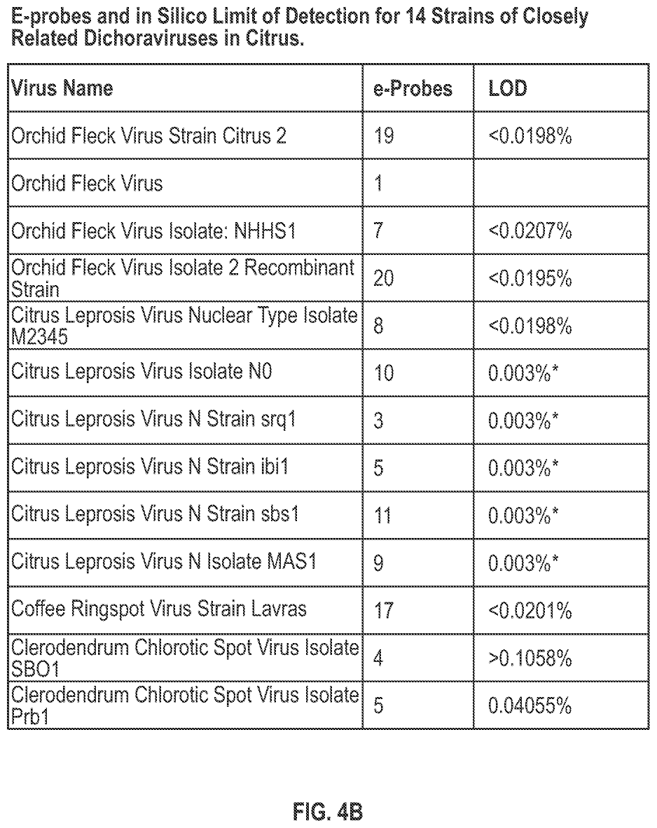

FIG. 4A illustrates a table of exemplary pathogens for grapes. For example, for grapes, grapevine pathogens may include a viral species comprised of a DNA virus, a viral species comprised of a (+)ssRNA virus, a bacterial pathogen of grapes, fungi pathogens of grapes, oomycetes of grapes, or the like as illustrated in FIG. 4A. To that end, the target genome 18 may be, for example, Grapevine Leafroll-associated Virus 3 (GLRaV-3). The target genomes 18 for each target pathogen may include all or a significant amount of separate genomes belonging to the taxonomy group of interest and acting as an inclusivity panel. Additionally, each target genome 18 for each target pathogen may include sequences from different geographical areas. FIG. 4B illustrates a table of exemplary pathogens for citrus, and in particular, e-probes designed for the detection of Dichoraviruses associated with citrus Leprosis disease syndrome. As shown in FIG. 4B, the table includes Dichoraviruses infecting citrus as target genomes 18 and host near-neighboring genomes 20 on Orchid, Hibiscus, Clerodendrum, and Coffee.

For determination of the raw e-probe 50, each target genome 18 may be associated with one or more near-neighbor genomes 20. The one or more near-neighbor genomes 20 act as an exclusionary panel. The one or more near-neighbor genomes 20 may include one or more organisms found in the taxonomy group of the target pathogen or taxonomically close relatives of the target pathogen to distinguish and contrast with the target genome 18. For example, in FIG. 4, the target genome 18 may include GLRaV-3 and the near-neighbor genomes 20 to that target genome 18 may include, for example, at least the remaining fourteen genomes listed within the table of exemplary pathogens for grapes.

Target genomes 18 and the one or more near-neighbor genomes 20 may comprise fully assembled genomes, substantially assembled genomes and/or draft genomes. In some embodiments, the target genome 18 may be provided as a collection of data stored in a first unit and the near-neighbor genome 20 may be provided as a collection of data stored in a second unit separate from the first unit. Each of the target genome 18 and the near-neighbor genome 20 may be stored in one or more database 42.

In some embodiments, the user may select a nucleotide (nt) length for each sequence of the e-probes 16 via the one or more external systems 34 and/or the input device 36 of the one or more processors 30. For example, the user may select the raw e-probes 50 to include between 20 nt to 120 nt. In some embodiments, the user may select the raw e-probes 50 to include between 20 nt to 60 nt for viruses and 60 nt to 100 nt for bacteria, fungi and oomycetes, for example.

In designing the raw e-probes 50, the processor 30 analyzes the target genome 18 and the one or more near-neighbor genomes 20 via a parallel comparison to generate the raw e-probes 50. Generally, the target genome 18 is compared to the one or more near-neighbor genome(s) 20 to find unique target sequence(s) of the target pathogen. The comparison may include identification of specific sequences of the target pathogen using a sequence alignment program that compares the target genome 18 with the one or more near-neighbor genomes 20. In some embodiments, the comparison may be determined via a whole genome alignment system, such as MUMmer, for example, to identify regions of similarity between the target genome 18 and the one or more near-neighbor genomes 20 to determine regions of unique target sequences for the target pathogen. In some embodiments, the parallel comparison may be via a k-mer based analysis system such that unique k-mers belonging solely to the target genome 18 may be determined. In some embodiments, global or local alignment tools may be used to identify similarities between the target genome 18 and the one or more near-neighbor genomes 20 to determine regions of unique target sequences for the target pathogen.

Similar sequences found between the target genome 18 and the one or more near-neighbor genomes 20 may be removed and unique sequences accepted as raw e-probes 50. For example, in FIG. 4, for the target pathogen GLRaV-3, a total of fifteen unique raw e-probes 50 were generated by the processor 30. The raw e-probes 50 are unique to the target pathogen.

Referring to FIG. 3, in the step 104, the raw e-probes 50 may be curated by eliminating one or more sequences having substantial similarities with other pathogens, hosts, and/or the like, to form curated e-probes 52. Curation of the raw e-probes 50 may include, eliminating raw e-probes 50 considered irrelevant to the target pathogen, specificity analysis of the sequence of the raw e-probes 50, and/or sensitivity analysis of the sequence of the raw e-probes 50.

Diagnostic sensitivity and/or specificity may be immediately adjusted during analysis by the user (e.g., probe developer) for fitness of purpose. Adjustability of diagnostic sensitivity and specificity immediately during analysis is unique and different from any other diagnostic assay method. Generally, via curation, diagnostic sensitivity and limit of detection (LOD) may be decreased while specificity is increased and vice versa. To that end, adjustability of diagnostic sensitivity and/or specificity during analysis is distinguishable to other diagnostic assays having mandated fixed values such as polymerase chain reaction (PCR) and enzyme-linked immunoassay (ELISA). Diagnostic sensitivity may be adjusted by increasing or decreasing the number of sequences included in an e-probe set. For example, to increase diagnostic sensitivity, curation of the raw e-probes 50 may allow for a greater number of curated e-probes 52 to be provided within an e-probe set based on one or more metrics (e.g., percent identity, alignment coverage, e-value). In contrast, to increase diagnostic specificity, raw e-probes 50 having relatively low percent identity or alignment coverage may be eliminated from an e-probe set.

Generally, during curation, raw e-probes 50 may be comparatively analyzed via a Basic Local Alignment Search Tool for nucleotides (BLASTn) from the National Center for Biotechnology Information (NCBI). Sequences may be analyzed using one or more database, including, but not limited to, a nucleotide database 60 (e.g., nt database compiled by NCBI), a protein database 62 (e.g., nr database compiled by NCBI), Reference Sequence database 64 (RefSeq), combinations thereof, and the like.

During comparative analysis, each raw e-probe 50 is compared with the one or more database (e.g., nt database 60, nr databases 62 and RefSeq database 64) and the host genome 66 to provide raw hits 70. Raw hits 70 are substantial matches to the sequence of the raw e-probe 50 with a minimum Eigenvalue (e-value). The e-value is a parameter that describes the number of substantial matches expected when searching a database of a particular size. The e-value may be used as an alignment metric to filter the raw e-probes 50 and is configured to be selected by the user (e.g., probe developer) based on fitness of purpose. For example, the user may select an e-value of 1×10⁻¹⁰ to provide a stringent analysis increasing diagnostic specificity. In another example, the user may select an e-value of 1×10¹ such that diagnostic sensitivity is increased.

Raw hits 70 analyzed during hit classification 72 determine if each raw e-probe 50 is a false positive e-probe 68 or a curated e-probe 52. Some raw e-probes 50 may cause false positive hits if there is spurious alignment with a sequence in another organism. For example, if the raw e-probe 50 substantially matches sequences other than the target pathogen (i.e., potential false positive), the raw hit 70 may be classified as a false positive e-probe 68 and eliminated from the dataset. In some embodiments, if the hit frequency of the raw e-probe 50 is determined to be greater than a pre-determined value, the raw hit 70 may be classified as a false positive e-probe 68 and the raw e-probe 50 is eliminated from the dataset. For example, if the raw e-probe 50 has a hit frequency higher than a predetermined value (e.g., 5), the raw hit 70 may be classified as a false positive e-probe 68 and eliminated from the data.

In some embodiments, the raw e-probes 50 may be comparatively analyzed with the host genome 66, and similarly, if the raw hit 70 substantially matches sequences within the host with a hit frequency above a predetermined value (e.g., 5), the raw hit 70 may be classified as a false positive e-probe and eliminated from the dataset. In some embodiments, if the raw hit 70 has an e-value lower than a pre-determined value and not from the target pathogen, the raw hit 70 may be classified as a false positive e-probe 68 and eliminated from the dataset. The remaining raw hits 70 may be considered curated e-probes 52.

In some embodiments, during curation, multiplicity analysis may be used to further curate the raw e-probes 50 to provide semi-quantitative e-probes 50, that are responsive to titer. Generally, multiplicity analysis (e.g., multiplying all hits per probe by −3, −1, 0, +1 or +3) may increase hit frequency for raw e-probes 50 that are responsive to titer and decrease hit frequency for raw e-probes 50 that are not responsive to titer. To that end, e-probes are ranked and raw e-probes not responsive to titer receive a hit classification 72 near zero and may then be removed from the dataset.

Referring to FIGS. 2-3, in the step 106, the e-probe design system 12 may provide one or more in silico validated e-probes 54 or in silico validated e-probe sets from the curated e-probes 52 via in silico validation. Generally, the curated e-probes 52 may undergo in silico validation with one or more simulated samples 82 and different ratios of the genome of the target pathogen to assess limit of detection (LOD), sensitivity and/or specificity. For example, in silico validation may determine theoretical sensitivity (i.e., true positive rate) and/or specificity (i.e., false positive rate) of the curated e-probe 52 using the one or more simulated samples 82. The LOD determines the lowest levels of the target pathogen that can be reliably detected using a scoring system. Based on the scoring system, curated e-probes 52 may be classified as in silico e-probes 54 or further eliminated from the dataset.

The one or more simulated samples 82 may be provided via a metagenome simulator 74. In particular, the one or more simulated samples 82 may be developed by creating one or more metagenomic simulations that include the host 76, a gradient of pathogen genomes 78, and related microbiome 80. In some embodiments, the metagenome simulator 74 may be provided within the processor 30. In some embodiments, the metagenome simulator 74 may be provided via one or more external systems 34. In some embodiments, the simulated samples 82 may be provided via high-throughput such as NanoSim, MetaSim, ART, and/or one or more type of high-throughput sequencing simulators. In some embodiments, simulated samples 82 may be capped (e.g., one million total reads).

The one or more simulated samples 82 may be provided to the processor 30 and compared with the curated e-probes 52 to determine a comparative hit. One or more alignment metrics may be predetermined by a user to classify the comparative hit as a positive hit or a negative hit. The one or more alignment metrics may include, but are not limited to, percent identity, query coverage of the comparative hit, and the like. The one or more alignment metrics may be selected to simulate high comparative hit stringency or low comparative hit stringency. A comparative score may be determined for each comparative hit based on the percent identity and query coverage. Scores are generated for each sequence of the curated e-probe 52. The probability that a comparative hit is positive or negative may be based on the comparative score. For example, percent identity and query coverage may be selected to be above 95% to classify a comparative hit as a positive hit. A positive comparative hit validates the curated e-probe 52 as an in silico validated e-probe 54. A negative comparative hit may eliminate the curated e-probe 52 from the dataset. By way of example, a 100% match for one curated e-probe 52 for the simulated sample of the target pathogen may appear as follows:

(SIMULATED SAMPLE)
(SEQ ID NO: 1)
AAATTGGCCGGCCTTACCCGG
(CURATED E-PROBE)
(SEQ ID NO: 2)
AAATTGGCCGGCCTTACCCGG

A 60% match for the curated e-probe for the simulated sample may appear as follows:

(SIMULATED SAMPLE)
(SEQ ID NO: 3)
AAATTGGCCGGCCTTACCCGG
(CURATED E-PROBE)
(SEQ ID NO: 4)
TAAATGGGCGGGCTTACCCGC

The comparative score is equal to E-Probe Hits x Percent match of each hit. In particular:

$$score = {\sum }_{j = 1}^{n} \frac{[ \frac{{p}_{j}}{100} + ( \frac{{a}_{j} - {g}_{j}}{L} ) ]}{2} \qquad ( EQ . 1 )$$

wherein n is number of hits that the e-probe sequence had with the HTS data; j is 1, 2, . . . n; p is alignment percent identity (e.g., 90 to 100 percent); a is alignment length (e.g., 35 to the maximum e-probe length;
g is gap length in the alignment; Lis the length of e-probe (e.g., 60 nt, 80 nt).

Equations 2-4 illustrate another exemplary comparative score for use with curated e-probes 52. In particular, EQ. 2 includes:

T=Σᵢ₋₁^{k}Sᵢ=Σᵢ₌₁^{k}PIᵢ×PCᵢ (EQ. 2)

wherein:

$${PI}_{i} = \frac{{n}_{i}}{{m}_{i}} \times 100 \% \qquad ( EQ . 3 )$$

$${PC}_{i} = \frac{{m}_{i}}{N} \times 100 \% \qquad ( EQ . 4 )$$

wherein PIᵢ is the percentage identity for E-probe i; PCᵢ is the percentage coverage for E-probe i and Sᵢ is the score for E-probe i, wherein i=1, 2, . . . , k, and k is number of E-probes; nᵢ is the number of matches of nucleotide of sequence in E-probe i; mᵢ is the number of total nucleotide in E-probe i; N is the number of total nucleotide in the metagenome; and, T is the total score.

The probability that the target pathogen is within the simulated sample 82 is generated using scores of known positive simulated samples 82 and negative simulated samples 82. The LOD is then the point at which there exists a 50/50 chance of a false negative. The LOD is thus the threshold for a positive or negative determination, and thus, acceptance of a validated e-probe or elimination of the e-probe from the dataset.

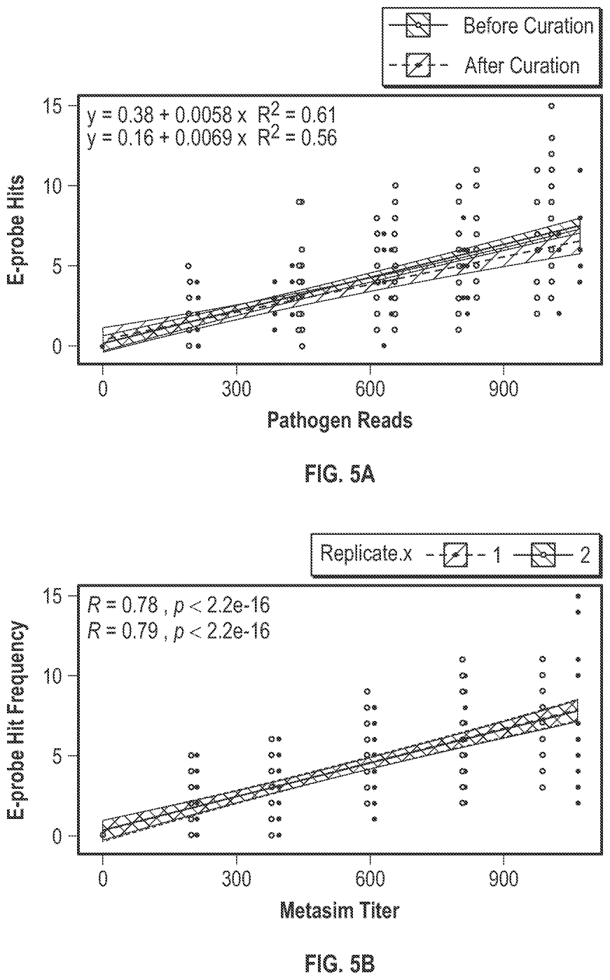

Referring to FIGS. 2-3 and 5A, using data from the in silico validation, a linear regression may be generated to illustrate theoretical sensitivity and limit of detection (LOD) at the intercept of the linear regression equation. FIG. 5A illustrates a linear comparison of raw e-probes 50 and curated e-probes 52 of GLRaV-3 before and after curation. Generally, LOD increases with curation of the raw e-probes 50. For example, before curation, the LOD of raw e-probes 50 of GLRaV-3 reached at 400 pathogen reads when evaluating fifteen raw e-probes 50. Curation leads to five curated e-probes 52. After curation, the limit of detection was increased to 600 pathogen reads. Curation also may improve quantitative capacity as observed in the R² difference between raw e-probes 50 and curated e-probes 52 shown in FIG. 5A. FIG. 5B illustrates another exemplary linear comparison using data from the in silico validation to illustrate theoretical sensitivity and LOD for e-probes of the Dichoraviruses illustrated in FIG. 4B in accordance with the present disclosure. The table in FIG. 4B provides the resulting LOD from analysis.

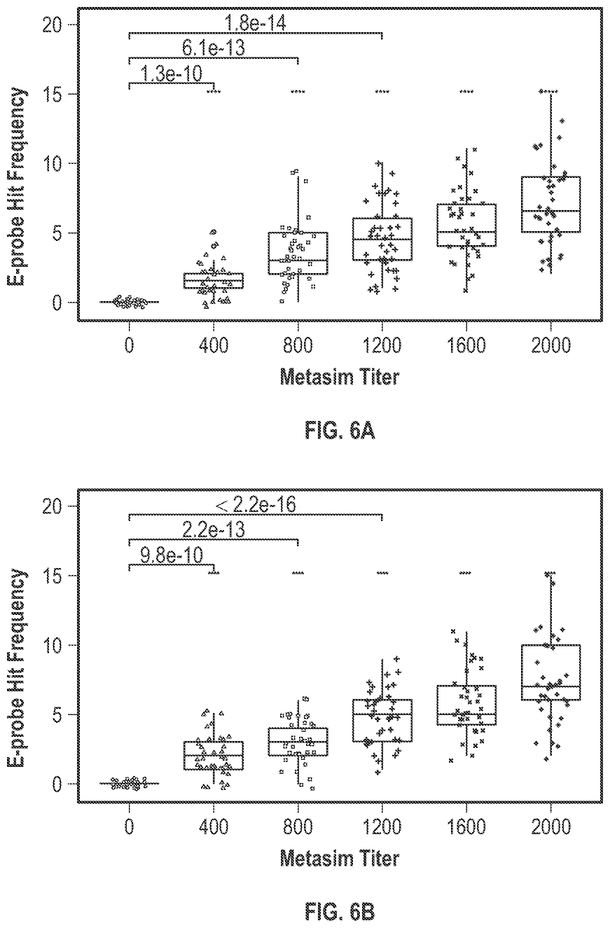

FIG. 6A illustrates a boxplot depicting pathogen titer response with fifteen curated e-probes 52 in-silico for GLRaV-3. Simulated samples of the grape genome and GLRaV-3 at various concentrations were provided for the example. The curated e-probes 52 were used and comparative hits determined. The boxplot depicts the hit distribution of the curated e-probes 52 and a known pathogen titer in the simulated sample 82 (shown in FIG. 3). As shown in FIG. 6A, the average comparative hits for the curated e-probes 52 decreased for each serial dilution of the pathogen. Curated e-probes 52 that are unresponsive to titer, that is the comparative hit frequency of the curated e-probe 52 does not increase in relation to abundance of the pathogen, may be identified and removed. The remaining curated e-probes 52 may be identified as validated e-probes or in silico validated e-probes 54. To that end, in silico validated e-probes 54 are determined by the curated e-probe(s) 52 most responsive to pathogen gradient or titer with response to pathogen titer being the number of times the curated e-probe 52 has a comparative hit (i.e., matching sequence to the simulated sample 82). FIG. 6B illustrates another exemplary boxplot depicting pathogen titer response with thirteen e-probes in-silico for Dichoraviruses (shown in FIG. 4B) in accordance with the present disclosure.

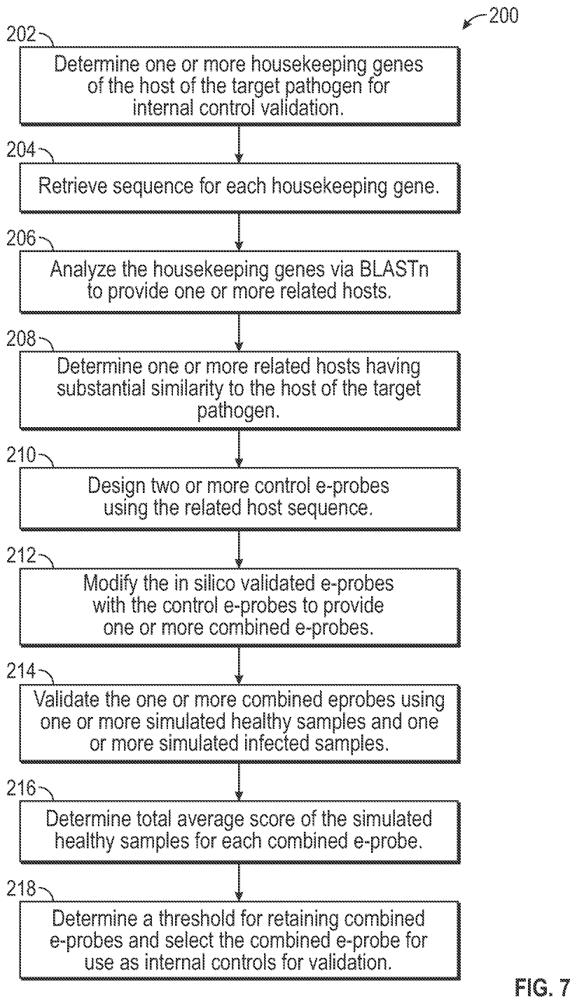

Referring to FIG. 7, in some embodiments, internal control e-probes may be designed to further validate the in silico validated e-probes 54. FIG. 7 illustrates a flow chart 200 of an exemplary method for determining and providing internal control e-probes for validation of the curated e-probes 52 and/or the in silico validated e-probes 54. In a step 202, one or more host genes that are highly conserved housekeeping genes may be determined for internal control validation. For example, for a citrus host, cytochrome oxidase 6, cytochrome oxidase 15 and NADH dehydrogenase 1 alpha subcomplex subunit may be used for internal control validation. In a step 204, sequences for the one or more housekeeping genes may be retrieved. For example, the one or more housekeeping genes may be retrieved from the NCBI database. In a step 206, sequences may be comparatively analyzed via a Basic Local Alignment Search Tool for nucleotides (BLASTn) from the National Center for Biotechnology Information (NCBI) to provide one or more similar hosts (for example, any other woody fruit or nut tree for citrus or any other flowering ornamental bush for roses). In a step 208, hosts having substantial similarity to the host of the target pathogen may be determined. For example, hosts having approximately 77% to 85% similarity to the citrus housekeeping genes were identified from perennial plants such as Prunus persica (prune trees), Pistacia vera (pistachio trees), and Malus domestica (apple trees). The percentage of similarity may be determined based on design considerations. In a step 210, a user may manually design two or more control e-probes using the related host sequences, with each control e-probe having different lengths. For example, three control e-probes having lengths of 20 nt, 30 nt and 40 nt may be designed. In a step 212, modify the in silico validated e-probes 54 by adding the internal control sequence e-probes to the combined e-probe set. In a step 214, using one or more simulated healthy samples (e.g., ten healthy samples) and one or more simulated infected samples (e.g., ten infected samples) validate each combined e-probe sets and determine a score for each comparative hit based on the percent identity and query coverage. In a step 216, total average score of the simulated healthy samples (e.g., negative control samples) for each combined e-probe may be determined to generate a non-zero variance for the quadratic discriminate analysis. For example, the total average score for each combined e-probe may be determined for each combined e-probe appears in at least 8 to 10 of the simulated healthy samples used. In a step 218, determine a threshold for retaining combined e-probes and select the combined e-probes for use as internal controls for validation. For example, the combined e-probes may be ranked from lowest to highest total average score and the top five lowest scoring combined e-probes may be retained for internal controls for validation. Internal controls provide a non-zero variance for quadratic discriminate analysis. Each e-probe set (e.g., curated e-probe set 52, in silico validated e-probe set 54) provided in the e-probe diagnostic system 14 may include internal control e-probes. The e-probe design system 12 generally uses at least five internal control e-probes for validation of curated e-probes 52 and/or in silico validated e-probes 54. Such informal control e-probes provide at least (1) an indication that extraction was successful; and, (2) provide a non-zero variance for the quadratic discriminate analysis in accordance with the present disclosure.

Referring to FIGS. 2 and 3, in the step 108, the e-probe design system 12 may provide in vivo or in vitro validated e-probes 56 from the curated e-probes 52 and/or the in silico validated e-probes 54 via in vitro validation. The in vitro validation is similar to in silico validation. In vitro samples 84 are used to analyze for diagnostic sensitivity 86 and/or diagnostic specificity 88 of the curated e-probes 52 and/or the in silico validated e-probes 54. In some embodiments, at least ten positive in vitro samples and at least ten negative in vitro samples may be used for in vitro validation. The processor 30, using techniques similar to the in silico validation, may determine limit of detection (LOD) as described herein. In some embodiments, in vitro validation may include use of in vitro samples spiked with a gradient of the target pathogen. Spiking may be at the organismal, cellular, or molecular nucleic acid level. The in vitro spiked sample may be analyzed for diagnostic sensitivity 86 and diagnostic specificity 88 using the curated e-probes 52 or in silico validated e-probes 54 to generate data related to sensitivity and LOD. Curated e-probes 52 or in silico validated e-probes 54 that are unresponsive to titer when using the in vitro samples, that is the hit frequency of the in silico validated e-probe 54 does not increase in relation to abundance of the pathogen in the in vitro sample, may be identified and removed with the remaining in silico validated e-probes 54 deemed as in vitro validated e-probes 56. To that end, in vitro validated e-probes 56 are determined to be the most responsive to pathogen gradient or titer with response to pathogen titer being the number of times the in silico validated e-probe 56 has a comparative hit (i.e., matching sequence to the simulated sample).

The LOD generally provides the lowest levels of target pathogen that may be reliably detected in the samples 82 by the in vitro or in vivo validated e-probes 56. Generally, the algorithm for LOD may be developed for a particular target pathogen. The algorithm is based on the Bayes decision boundary and developed using mean and variance of positive and negative samples 82. The algorithm for LOD is based on the probability that the target pathogen is positive or negative in the sample 82 and is determined using the comparative scores for the samples 82. Equation 5 is an exemplary algorithm for LO D.

$$LOD = x = \frac{( \frac{{μ}_{2}}{{σ}_{2}^{2}} - \frac{{μ}_{1}}{{σ}_{1}^{2}} ) - \sqrt{\frac{{( {μ}_{1} - {μ}_{2} )}^{2}}{{σ}_{1}^{2} {σ}_{2}^{2}} - ( \frac{1}{{σ}_{2}^{2}} - \frac{1}{{σ}_{1}^{2}} ) \times 2 log \frac{{σ}_{2}}{{σ}_{1}}}}{( \frac{1}{{σ}_{2}^{2}} - \frac{1}{{σ}_{1}^{2}} )} \qquad ( EQ . 5 )$$

wherein μ₁ is the mean score of the positive samples, μ₂ is the mean score of the negative samples; and σ₁ is the variance of the positive sample, and σ₂ is the variance of the negative sample. The algorithm tested with known positive and negative metagenomic sequence data of the target pathogen, determines the LOD of the relevant e-probe set. It should be noted internal control sequences assure a non-zero variance in the negative control.

Referring to FIGS. 2 and 3, in the step 110, the in silico validated e-probes 54 and/or the in vitro validated e-probes 56 may be field validated to provide field validated e-probes 58. For field validation, known field samples 90 having positive pathogen symptoms and negative pathogen symptoms, ranging from asymptomatic to highly symptomatic, may be sequenced 92. Results for field validation may be compared against a known standard assay for verification (e.g., PCR, ELISA) and in the case of false positive, in vitro validated e-probes 56 that are hitting may be eliminated.

Verified curated e-probes 52, in silico validated e-probes 54 and/or in vitro validated e-probes 56 may be stored in one or more database 42 as the e-probe 16 for use by the interactive pathogen detection system 10 (e.g., pathogen detection). In some embodiments, metadata crediting developer and/or institution of development of the e-probe 16, description of the level of validation (e.g., curated, in silico validation, in vitro validation, field validation), publications relating to the e-probe 16, and the like, may be stored in the one or more database 42.

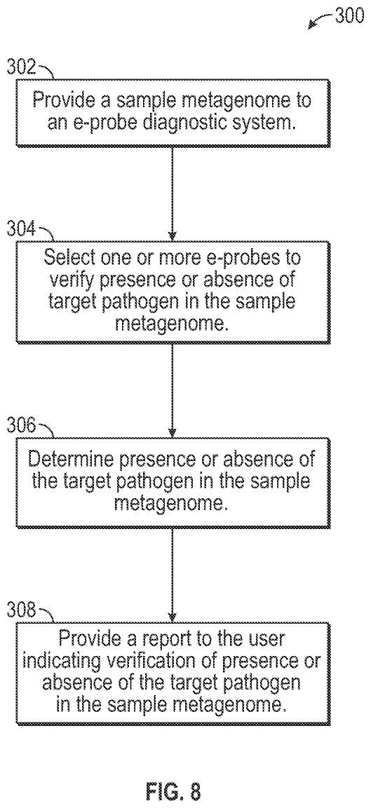

Referring to FIGS. 1 and 8, e-probes 16 may be used for detection of one or more target pathogens in the sample metagenomes 22 provided to the e-probe diagnostic system 14. The e-probe diagnostic system 14 provides testing for target pathogens simultaneously rather than sequentially. That is, the e-probe diagnostic system 14 is configured to test for all pathogens of concern in a single test on a single sample metagenome 22. Further, testing of the sample metagenome 22 does not require isolation of the target pathogen(s), amplification of the signature of the target pathogen(s), genomic or transcriptomic assembly, or other resource intensive protocols.

FIG. 8 illustrates a flow chart 300 of an exemplary method of detecting one or more target pathogens in the sample metagenome 22 using e-probes 16. In a step 302, a user may provide the sample metagenome 22 to the e-probe diagnostic system 14. The sample metagenome 22 may include sequencing of a plant specimen containing microbes and pathogens, for example. For animal disease diagnostics, a tissue sample or swab may be sequenced.

In some embodiments, the e-probe diagnostic system 14 may include a sequence calculator 98. The sequence calculator 98 indicates the amount of sequencing of the sample metagenome 22 needed to find the target pathogen. Equation 6 provides an exemplary algorithm for use in the sequence calculator 98.

$$k - n \frac{a}{a + b} - \sqrt{n \frac{a}{a + b}} {z}_{1 - p} = 0 \qquad EQ . 6$$

wherein k is the number of reads desired to detect; n is the average read length (normal distribution); a is the pathogen genome size; b is the host genome size; and, p is the probability. The sequence calculator 98 may allow the user to limit sequencing depth of the sample metagenome 22 to preserve sequencing flow cell for more samples and thus reduce cost.

In a step 304, the user may select e-probes or e-probe sets to verify presence or absence of one or more target pathogen in the sample metagenome 22. In a step 306, the e-probe diagnostic system 14 may determine presence or absence of the one or more target pathogens in the sample metagenome 22 using the e-probes 16 or e-probe sets. The e-probe diagnostic system 14 compares the sequence of the e-probe 16 to the sample metagenome 22. A threshold for positive detection may be pre-determined. If the threshold for positive detection is reached, the e-probe diagnostic system 14 determines presence of the target pathogen in the sample metagenome 22. The threshold may be a fixed scoring number, such as the p-value, for example, obtained from validation or statistical analysis with the unknown sample versus a known negative control. In using the p-value, for example, the statistical comparison with the unknown sample and the known negative control generates a p-value, if the p-value is at 0.05 or below, the unknown sample may be considered positive.

In some embodiments, the presence or absence of the one or more target pathogens in the sample metagenome 22 may be determined in seconds. In some embodiments, the presence or absence of multiple target pathogens in the sample metagenome 22 may be determined in seconds. In some embodiments, the presence or absence of the one or more target pathogens in the sample metagenome 22 may be determined in minutes. In some embodiments, the presence or absence of multiple target pathogens in the sample metagenome 22 may be determined in minutes. In a step 308, the e-probe diagnostic system 14 may provide a report to the user. The report may indicate verification of presence or absence of the target pathogen in the sample metagenome 22. In some embodiments, the report may contain additional treatment options including, but not limited to, therapeutic treatment, prophylactic and/or preventative measures related to the target pathogen.

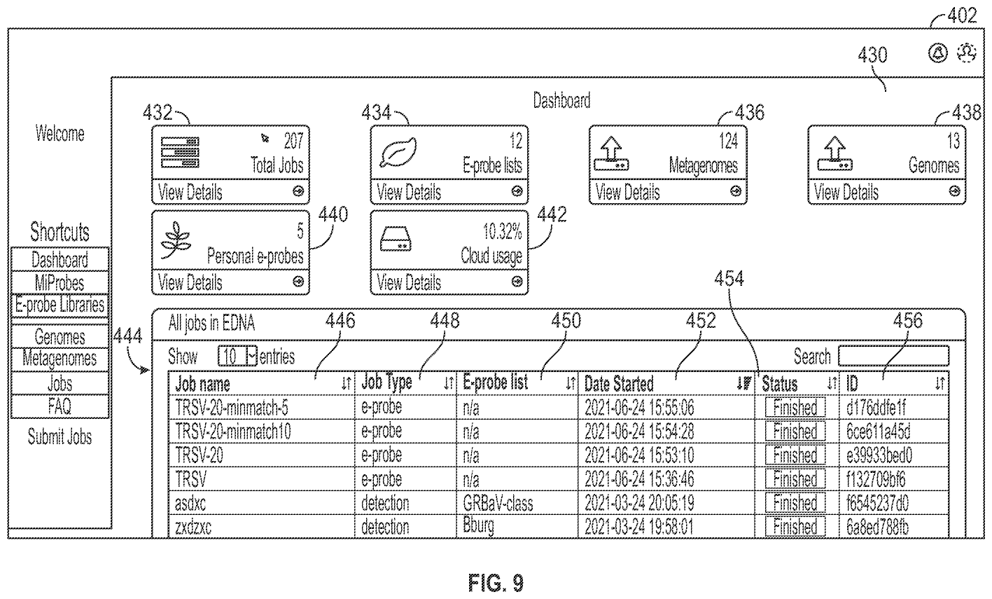

FIGS. 9-18 illustrate exemplary screenshots of an interactive pathogen detection system 10. Generally, a user may interact with the e-probe design system 12 and the e-probe diagnostic system 14 via a graphical user interface (e.g., via web page, network page, local page). The user interface may be used to change values within one or more properties, upload documents, and the like. The user interface may be provided via the processor 30 and/or external systems 34 as described herein in relation to FIG. 2.

FIGS. 9-12 illustrate exemplary screenshots 400, 402, 404 and 406 directed to the e-probe design system 12. FIG. 9 illustrates an exemplary screenshot 400 of a dashboard 430 for the e-probe design system 12. The dashboard 430 includes links including, but not limited to, job link 432, e-probe link 434, metagenome link 436, genome link 438, personal e-probe link 440, cloud memory usage link 442, and the like. As an example, a user may view the job link 432 as shown below the dashboard. The job link 432 provides a job listing 444 of all current and past jobs wherein a job is a design of at least one e-probe 16 (shown in FIG. 1). Field of the job listing 444 may include job name 446, job type 448 (e.g., e-probe design or e-probe detection), e-probe used 450 (for an e-probe detection job), initiation date 452, status 454, an assigned identification number (ID) 456, combinations thereof, and the like. The e-probe link 434 may provide an e-probe listing of current e-probes for use in the interactive pathogen detection system 10 with the personal e-probe link 440 providing a listing of e-probes developed specifically by the user. The metagenome link 436 may provide a listing of sample metagenomes 22 for use in the e-probe diagnostic system 14. The cloud memory usage link 442 provides details on the amount of memory allowed for the particular user.

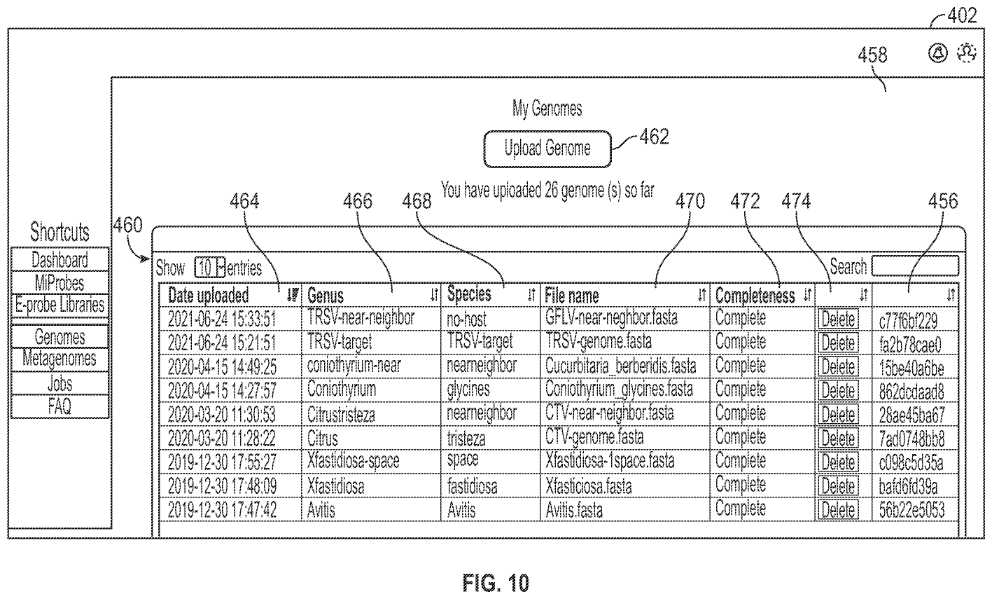

FIG. 10 illustrates an exemplary screenshot 402 of the genome site 458. The genome site 458 provides a genome listing 460 and an upload link 462. Referring to FIGS. 2-3 and 10, the upload link 462 allows the user to provide to the processor 30 at least one target genome 18 and at least one near-neighbor genome 20. Once uploaded, the at least one target genome 18 and the at least one near-neighbor genome 20 are provided to the genome listing 460. The genome listing 460 includes fields for upload date 464, genome type 466 (target or near-neighbor), host type 468, file name 470, status 472, assigned identification number (ID) 456, delete option 474, combinations thereof, and the like.

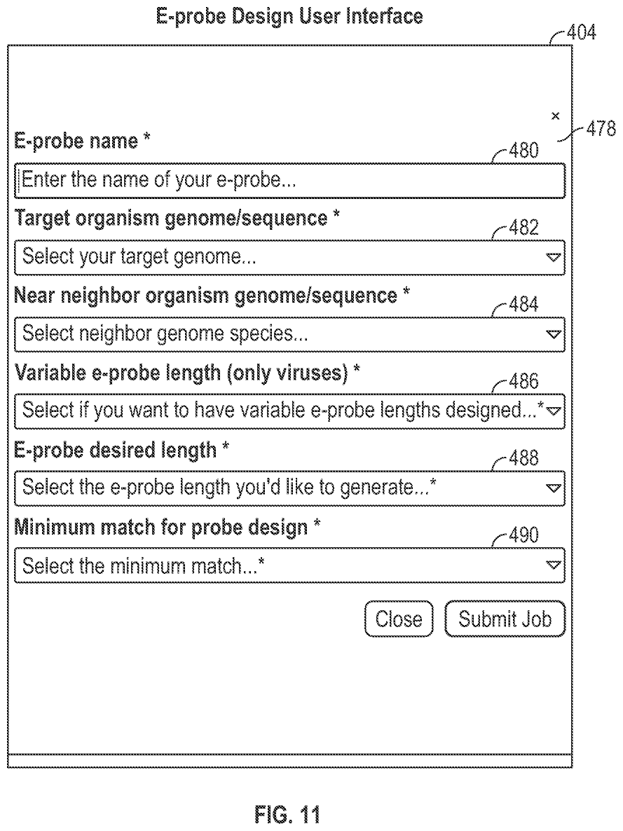

FIG. 11 illustrates an exemplary screenshot 404 of job submission 478. The user is able to select a name of the e-probe design in a name field 480. The user is able to select the target genome 18 from the target genome field 482 and the near-neighbor genome 20 from the near neighbor field 484. In some embodiments, the user may select whether to provide for a variable e-probe length or a fixed e-probe length in the variable field 486. The user is also able to select a desired e-probe length (e.g., 20 nt, 40 nt, 60 nt, 80 nt, 120 nt) in the length field 488. The minimum allowed match for the e-probe design (e.g., 15 matches) may be selected in the match field 490.

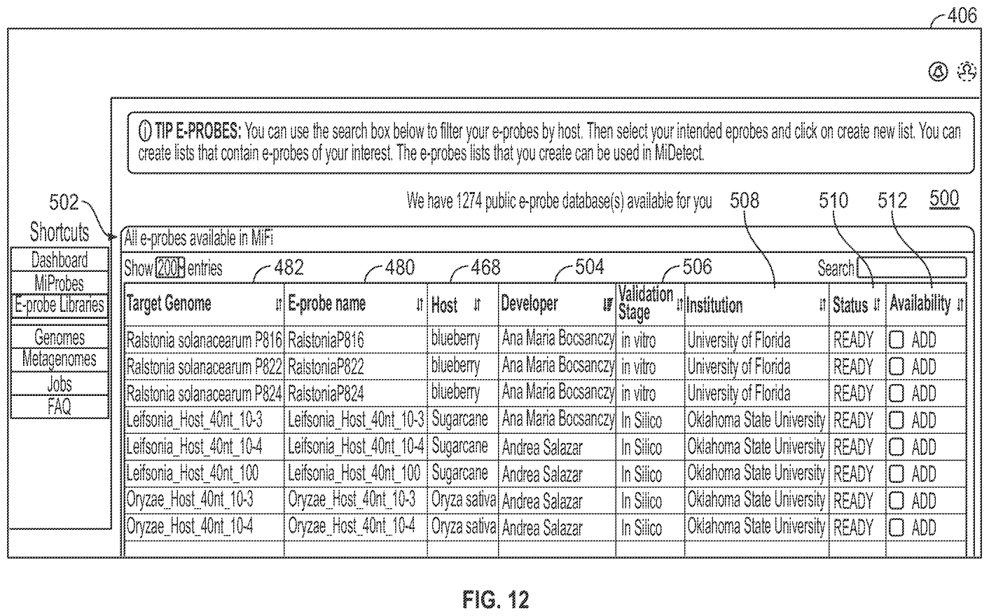

FIG. 12 illustrates an exemplary screenshot 406 of an e-probe library 500. The e-probe library 500 provides a listing 502 of e-probes 16 available to a user subsequent to design of e-probes 16 by the user in accordance with the present disclosure. Additionally, the listing 502 includes e-probes 16 publicly available for use by the user (e.g., use in the e-probe diagnostic system 14). The listing 502 includes the target genome field 482, name field 480, host type 468, developer 504, validation stage 506, institution of development 508, status 510, availability field 512, combinations thereof, and the like. The developer 504 and the institution of development 508 may identify the origin of the design of the e-probe 16. The validation stage 506 indicates the current stage of the e-probe (e.g., curated e-probe 52, in silico validated e-probe 54, in vitro validated e-probe 56, field validated e-probe 58). The status 510 of the e-probe 16 indicates if the e-probe 16 is currently ready to be used in the e-probe diagnostic system 14. If the e-probe is currently ready to be used in the e-probe diagnostic system 14, the availability field 512 may be selected to add the e-probe 16 for testing.

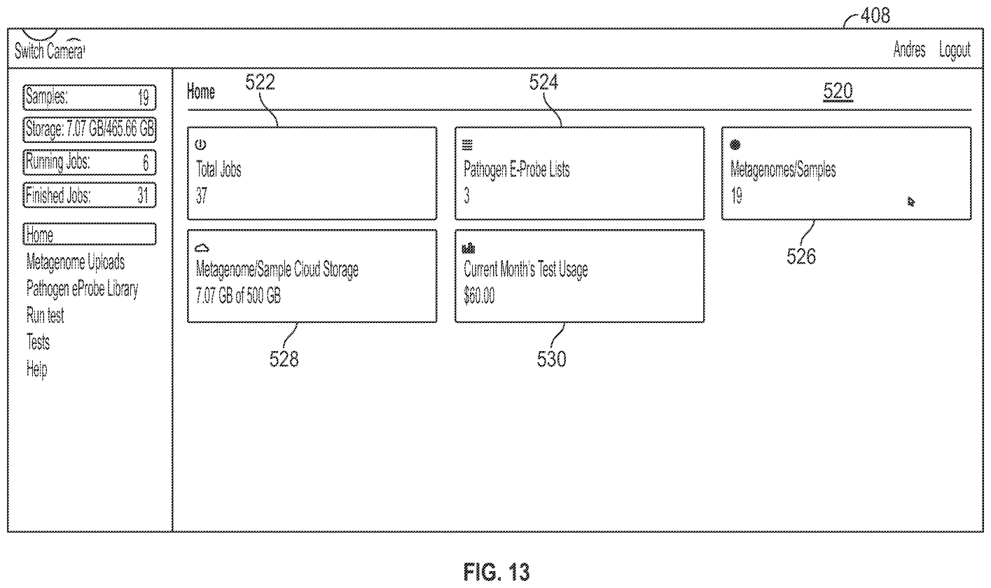

FIGS. 13-18 illustrate exemplary screenshots 408, 410, 412, 414, 416 and 418 directed to the e-probe diagnostic system 14. FIG. 13 illustrates an exemplary screenshot 408 of a dashboard 520 of the e-probe diagnostic system 14. The dashboard 520 includes links including, but not limited to, job link 522, pathogen e-probe list link 524, metagenome link 526, cloud memory usage link 528, current usage link 530, and the like. The job link 522 provides a job listing of all current and past jobs wherein a job the determination of the presence or absence of one or more pathogens and/or one or more microbes in a sample metagenome 22 using e-probes 16 (shown in FIG. 1). The pathogen e-probe list link 524 may provide an e-probe library of current e-probes for use in the interactive pathogen detection system 10. The metagenome link 526 may provide a listing of sample metagenomes 22 for use in the e-probe diagnostic system 14. The cloud memory usage link 528 provides details on the amount of memory allowed for the particular user. The current usage link 530 may provide details on usage of the user, payment plans of use of the e-probe diagnostic system 14, and the like.

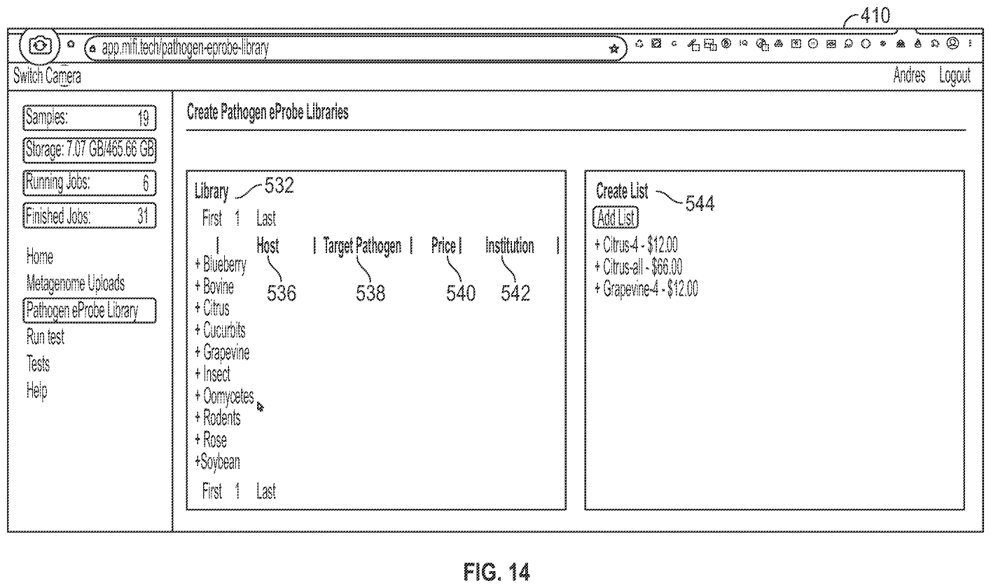

FIG. 14 illustrates an exemplary screenshot 410 of an exemplary e-probe library 532 for use in the e-probe diagnostic system 14. E-probes 16 within the e-probe library 532 may be designed in accordance with the present disclosure. The e-probe library 532 provides a listing 534 of available e-probes 16. The listing 534 may be distributed by genus type and provide fields such as a host field 536, target pathogen field 538, price point field 540, institution 542, and the like. The user is able to add e-probes 16 to a creation list 544 for use in the e-probe diagnostic system 14. The creation list 544 allows for e-probes 16 to be used for determination of the presence or absence of one or more pathogens and/or one or more microbes in a sample metagenome 22. Each e-probe 16 may be assigned a monetary value for use in the e-probe diagnostic system 14. For example, as shown in FIG. 14, the e-probe 16 for Citrus-4 is assigned a monetary value of $12.00 for use in the e-probe diagnostic system 14.

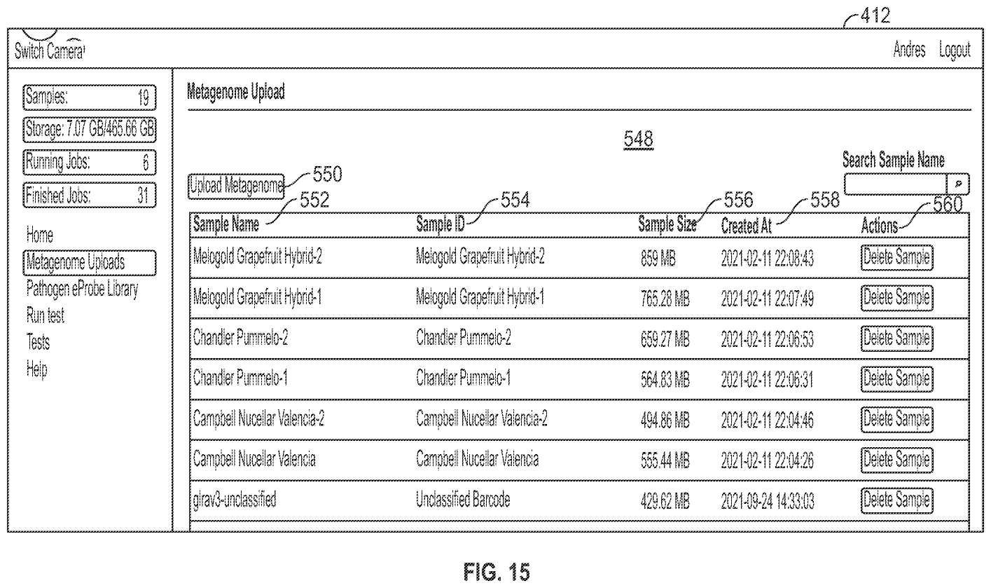

FIG. 15 illustrates a screenshot 412 of an exemplary metagenomic sequence listing 548. The metagenomic sequence listing 548 includes an upload option button 550 to allow a user to upload one or more sample metagenomes 22 for testing in the e-probe diagnostic system 14. The metagenomic sequence listing 548 may include fields such as the metagenomic sample name 554, a sample identification (ID) tag 556, sample size 558, creation date 560, deletion option field 562, combinations thereof, and the like.

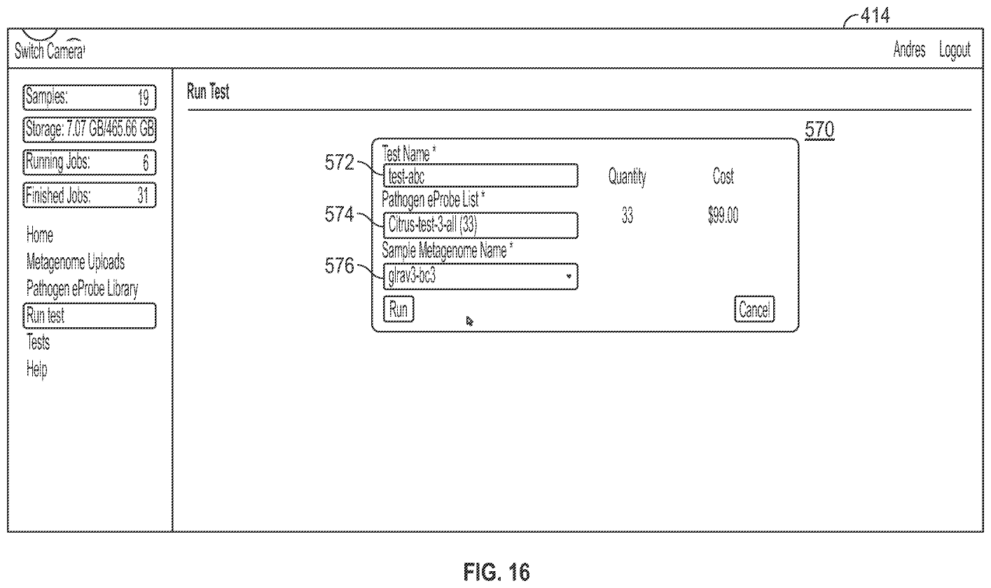

FIG. 16 illustrates a screenshot 414 of an exemplary test run site 570 for using e-probes 16 to determine presence or absence of one or more pathogens and/or one or more microbes in a sample metagenome 22. The test run site 570 may include a test name field 572, a pathogen e-probe list 574, and a sample metagenomic field 576. The test name field 572 may be selected by a user to distinguish between different tests. The pathogen e-probe list 574 is compiled from the creation list 544 shown in FIG. 14. In some embodiments, the pathogen e-probe list 574 may indicate the number of e-probes 16 being used for the particular test and the associated cost as shown in FIG. 16. The sample metagenomic field 576 may allow a user to select the sample metagenome 22 from the metagenomic sequence listing 548 shown in FIG. 15.

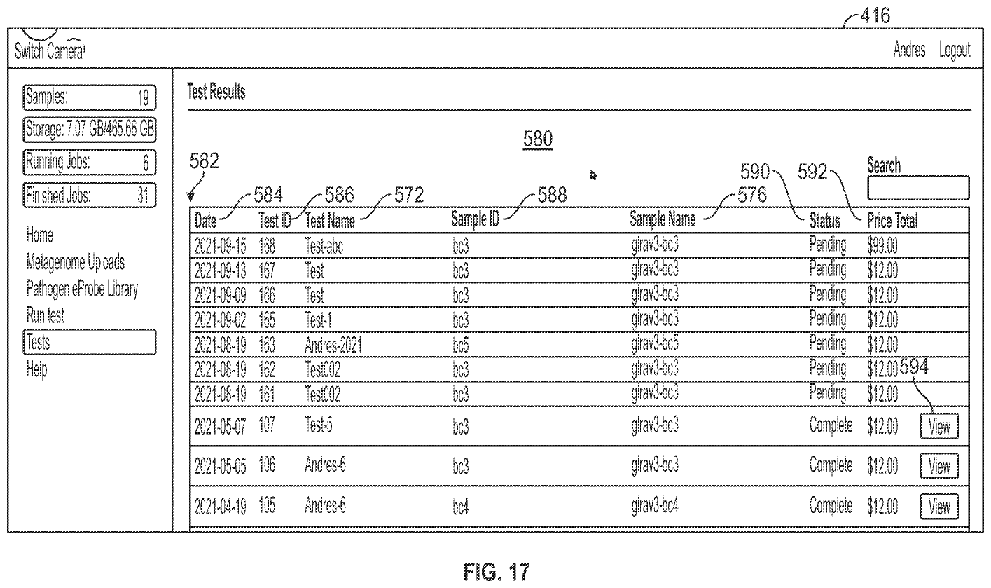

FIG. 17 illustrates a screenshot 416 of an exemplary comprehensive test results site 580 for the e-probe diagnostic system 14. The test results site 580 may include a test results listing 582 having fields such as a date field 584, test ID field 586, test name field 572, sample ID 588, sample metagenomic field 576, status field 590, and a total price field 592. Additionally, the test results listing 582 may provide an option button 594 for viewing a completed test.

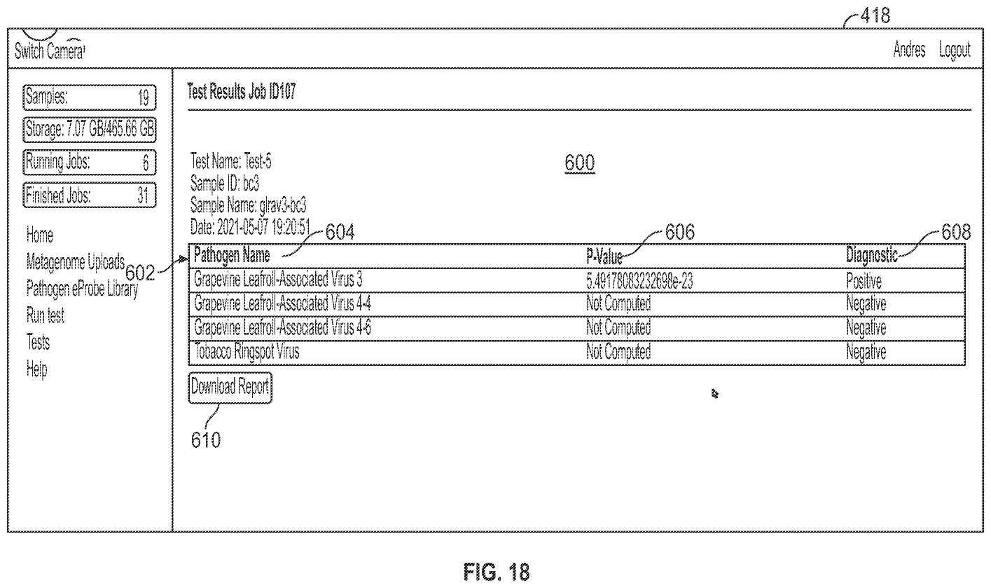

FIG. 18 illustrates a screenshot 418 of an exemplary completed test results site 600 for an individual test. The completed test results site includes a job listing 602 having fields such as a pathogen name field 604, a p-value field 606, and a diagnostic field 608. The pathogen name field 604 provides the listing of target pathogens for the individual test with the associated p-value field 606 when the diagnostic test is performed by the e-probe diagnostic system 14 for the particular sample. The diagnostic field 608 provides the determination of the presence (positive) or absence (negative) of one or more pathogens and/or one or more microbes in the particular sample by identification via the e-probes 16. The user may download one or more reports via the download report button 610.

The following is a number list of non-limiting illustrative embodiments of the inventive concept disclosed herein:

1. A method, comprising: receiving, by a processor, at least one target genome file, the target genome file including a genome sequence of a target pathogen; receiving, by a processor, at least one near-neighbor genome file, the near-neighbor genome file including a genome sequence of at least one organism found in a taxonomy close relative of the target pathogen; analyzing the target genome file and the near-neighbor genome file via a parallel comparison to generate a plurality of raw e-probe sequences to provide at least one raw e-probe sequence set, with each raw e-probe sequence set unique to the target pathogen; curating the plurality of raw e-probes sequences to classify each raw e-probe as a curated e-probe or a false positive e-probe, the curated e-probes forming at least one curated e-probe sequence set; performing in silico validation on the at least one curated e-probe sequence set to provide an in silico validated e-probe set, in silico validation including the steps of: obtaining at least one simulated sample provided by a metagenome simulator, the at least one simulated sample having different relative prevalence of the genome sequence of the target pathogen mixed into host genome sequences; determining comparative hits between the at least one curated e-probe sequence set and the at least one simulated sample; classifying the comparative hits using at least one alignment metric; validating the curated e-probe sequence set as the in silico validated e-probe set based on the classification of the comparative hits; and, determining, by an e-probe diagnostic system, presence of the target pathogen in a sample metagenome of a host using the in silico validated e-probe set.

2. The method of the illustrative embodiment 1, wherein the target genome file includes a partially assembled genome sequence of the target pathogen.

3. The method of illustrative embodiment 1, wherein the target genome file includes a draft subset genome of the target pathogen.

4. The method of any one of illustrative embodiments 1-3, further comprising the step of selecting, by a user, nucleotide (nt) length for each raw e-probe.

5. The method of any one of illustrative embodiments 1-4, wherein curating the plurality of raw e-probe sequences adjusts diagnostic sensitivity of the curated e-probe sequence set.

6. The method of any one of illustrative embodiments 1-5, further comprising the step of performing in vitro validation on the at least one in silico validated e-probe set to provide an in vitro validated e-probe set, the in vitro validated e-probe set being used to determine presence of the target pathogen in a sample metagenome.

7. The method of illustrative embodiment 6, wherein performing in vitro validation on the curated e-probe sequence set to provide an in vitro validated e-probe set includes the steps of: providing a plurality of in vitro samples having the target pathogen; analyzing the plurality of in vitro samples with the at least one in silico validated e-probe set to determine at least one comparative hit; classifying the comparative hits using at least one alignment metric to determine a comparative score; and, validating the in silico validated e-probe set based on the comparative score to provide the in vitro validated e-probe set.

8. The method of any one of illustrative embodiments 6 or 7, further comprising the step of performing field validation on the in vitro validated e-probe set to provide a field validated e-probe set, the field validated e-probe set being used to determine presence of the target pathogen in a sample metagenome.

9. The method of any one of illustrative embodiments claim 1-8, further comprising the step of performing field validation on the in silico validated e-probe set to provide a field validated e-probe set, the field validated e-probe set being used to determine presence of the target pathogen in a sample metagenome.

10. The method of any one of illustrative embodiments 1-9, wherein curating the plurality of raw e-probe sequences includes comparative analysis of the raw e-probe sequences using a Basic Local Alignment Search Tool for nucleotides (BLASTn) and at least one database to provide the curated e-probe sequence set.

11. The method of illustrative embodiment 10, wherein curating the plurality of raw e-probe sequences further comprises performing a multiplicity analysis using p-values to eliminate non-responsive e-probes.

12. The method of any one of illustrative embodiments 1-11, wherein the at least one alignment metric includes percent identity and query coverage of the comparative hits.

13. The method of any one of illustrative embodiments 1-12, further comprising the step of validating the in silico validated e-probe set using internal control e-probes.

14. The method of illustrative embodiment 13, wherein validating the in silico validated e-probe set uses at least five internal control e-probes.

15. One or more non-transitory computer readable medium storing a set of computer executable instructions for running on one or more processors that when executed cause the one or more processors to: receive at least one target genome file and at least one near-neighbor genome file; analyze the target genome file and the near-neighbor genome file to generate a plurality of raw e-probes with each raw e-probe unique to a target pathogen; curate the plurality of raw e-probes to provide a curated e-probe set; receive at least one simulated sample and perform in silico validation on the curated e-probe set to provide an in silico validated e-probe set; and, determine presence of the target pathogen in a sample metagenome using the in silico validated e-probe set in an e-probe diagnostic system.

16. The one or more non-transitory computer readable medium storing a set of computer executable instructions for running on one or more processors of illustrative embodiment 15, wherein the one or more processors curate the plurality of raw e-probes by performing a multiplicity analysis using p-values to eliminate non-responsive e-probes.

17. The one or more non-transitory computer readable medium storing a set of computer executable instructions for running on one or more processors of illustrative embodiments 15 or 16, wherein in silico validation includes the steps of: providing at least one simulated sample from a metagenomic database, the simulated sample having different relative prevalence of a genome sequence of the target pathogen mixed into host genome sequences; analyzing the at least one simulated sample with the curated e-probe set to determine comparative hits; classifying the comparative hits using at least one alignment metric to determine a comparative score; and, validating the curated e-probe based on the comparative score to provide the in silico validated e-probe set.

18. The one or more non-transitory computer readable medium storing a set of computer executable instructions for running on one or more processors of illustrative embodiment 17, wherein the at least one alignment metric includes percent identity and query coverage of the comparative hits.

19. The one or more non-transitory computer readable medium storing a set of computer executable instructions for running on one or more processors of any one of illustrative embodiments 17 or 18, further comprising the step of validating the in silico validated e-probe set using internal control e-probes.

20. A method, comprising: receiving at least one target genome file and at least one near-neighbor genome file; analyzing the target genome file and the near-neighbor genome file to generate a plurality of raw e-probes unique to a target pathogen having a pathogen genome, each raw e-probe having a unique nucleic acid signature sequence selected from along a length of the pathogen genome; curating the plurality of raw e-probes to provide a curated e-probe set; receiving at least one simulated sample and perform in silico validation on the curated e-probe set to provide an in silico validated e-probe set; performing in vitro validation on the in silico validated e-probe set to provide an in vitro validated e-probe set, the in vitro validated e-probe set being used to determine presence of the target pathogen in a sample metagenome; and, determining presence of the target pathogen in a sample metagenome using the in vitro validated e-probe set in an e-probe diagnostic system.

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

What is claimed is:

1. A method, comprising:
receiving, by a processor, at least one target genome file, the target genome file including a genome sequence of a target pathogen;
receiving, by a processor, at least one near-neighbor genome file, the near-neighbor genome file including a genome sequence of at least one organism found in a taxonomy close relative of the target pathogen;
analyzing the target genome file and the near-neighbor genome file via a parallel comparison to generate a plurality of raw e-probe sequences to provide at least one raw e-probe sequence set, with each raw e-probe sequence set unique to the target pathogen;
curating the plurality of raw e-probes sequences to classify each raw e-probe as a curated e-probe or a false positive e-probe, the curated e-probes forming at least one curated e-probe sequence set;
performing in silico validation on the at least one curated e-probe sequence set to provide an in silico validated e-probe set, in silico validation including the steps of:
obtaining at least one simulated sample provided by a metagenome simulator, the at least one simulated sample having different relative prevalence of the genome sequence of the target pathogen mixed into host genome sequences;
determining comparative hits between the at least one curated e-probe sequence set and the at least one simulated sample;
classifying the comparative hits using at least one alignment metric;
validating the curated e-probe sequence set as the in silico validated e-probe set based on the classification of the comparative hits; and,
determining, by an e-probe diagnostic system, presence of the target pathogen in a sample metagenome of a host using the in silico validated e-probe set.

2. The method of claim 1, wherein the target genome file includes a partially assembled genome sequence of the target pathogen.

3. The method of claim 1, wherein the target genome file includes a draft subset genome of the target pathogen.

4. The method of claim 1, further comprising the step of selecting, by a user, nucleotide (nt) length for each raw e-probe.

5. The method of claim 1, wherein curating the plurality of raw e-probe sequences adjusts diagnostic sensitivity of the curated e-probe sequence set.

6. The method of claim 1, further comprising the step of performing in vitro validation on the at least one in silico validated e-probe set to provide an in vitro validated e-probe set, the in vitro validated e-probe set being used to determine presence of the target pathogen in a sample metagenome.

7. The method of claim 6, wherein performing in vitro validation on the curated e-probe sequence set to provide an in vitro validated e-probe set includes the steps of:
providing a plurality of in vitro samples having the target pathogen;
analyzing the plurality of in vitro samples with the at least one in silico validated e-probe set to determine at least one comparative hit;
classifying the comparative hits using at least one alignment metric to determine a comparative score; and,
validating the in silico validated e-probe set based on the comparative score to provide the in vitro validated e-probe set.

8. The method of claim 6, further comprising the step of performing field validation on the in vitro validated e-probe set to provide a field validated e-probe set, the field validated e-probe set being used to determine presence of the target pathogen in a sample metagenome.

9. The method of claim 1, further comprising the step of performing field validation on the in silico validated e-probe set to provide a field validated e-probe set, the field validated e-probe set being used to determine presence of the target pathogen in a sample metagenome.

10. The method of claim 1, wherein curating the plurality of raw e-probe sequences includes comparative analysis of the raw e-probe sequences using a Basic Local Alignment Search Tool for nucleotides (BLASTn) and at least one database to provide the curated e-probe sequence set.

11. The method of claim 10, wherein curating the plurality of raw e-probe sequences further comprises performing a multiplicity analysis using p-values to eliminate non-responsive e-probes.

12. The method of claim 1, wherein the at least one alignment metric includes percent identity and query coverage of the comparative hits.

13. The method of claim 1, further comprising the step of validating the in silico validated e-probe set using internal control e-probes.

14. The method of claim 13, wherein validating the in silico validated e-probe set uses at least five internal control e-probes.

15. One or more non-transitory computer readable medium storing a set of computer executable instructions for running on one or more processors that when executed cause the one or more processors to:
receive at least one target genome file and at least one near-neighbor genome file;
analyze the target genome file and the near-neighbor genome file to generate a plurality of raw e-probes with each raw e-probe unique to a target pathogen;
curate the plurality of raw e-probes to provide a curated e-probe set;
receive at least one simulated sample and perform in silico validation on the curated e-probe set to provide an in silico validated e-probe set; and,
determine presence of the target pathogen in a sample metagenome using the in silico validated e-probe set in an e-probe diagnostic system.

16. The one or more non-transitory computer readable medium storing a set of computer executable instructions for running on one or more processors of claim 15, wherein the one or more processors curate the plurality of raw e-probes by performing a multiplicity analysis using p-values to eliminate non-responsive e-probes.

17. The one or more non-transitory computer readable medium storing a set of computer executable instructions for running on one or more processors of claim 15, wherein in silico validation includes the steps of:
providing at least one simulated sample from a metagenomic database, the simulated sample having different relative prevalence of a genome sequence of the target pathogen mixed into host genome sequences;
analyzing the at least one simulated sample with the curated e-probe set to determine comparative hits;
classifying the comparative hits using at least one alignment metric to determine a comparative score; and,
validating the curated e-probe based on the comparative score to provide the in silico validated e-probe set.

18. The one or more non-transitory computer readable medium storing a set of computer executable instructions for running on one or more processors of claim 17, wherein the at least one alignment metric includes percent identity and query coverage of the comparative hits.

19. The one or more non-transitory computer readable medium storing a set of computer executable instructions for running on one or more processors of claim 17, further comprising the step of validating the in silico validated e-probe set using internal control e-probes.

20. A method, comprising:
receiving at least one target genome file and at least one near-neighbor genome file;
analyzing the target genome file and the near-neighbor genome file to generate a plurality of raw e-probes unique to a target pathogen having a pathogen genome, each raw e-probe having a unique nucleic acid signature sequence selected from along a length of the pathogen genome;
curating the plurality of raw e-probes to provide a curated e-probe set;
receiving at least one simulated sample and perform in silico validation on the curated e-probe set to provide an in silico validated e-probe set;
performing in vitro validation on the in silico validated e-probe set to provide an in vitro validated e-probe set, the in vitro validated e-probe set being used to determine presence of the target pathogen in a sample metagenome; and,
determining presence of the target pathogen in a sample metagenome using the in vitro validated e-probe set in an e-probe diagnostic system.


SEQUENCE LISTING (4 sequences)

SEQ ID NO: 1 - DNA, 21 bp, synthetic construct
  misc_feature 1..21: note = Simulated Sample
  source 1..21: mol_type = other DNA
aaattggccggccttacccgg

SEQ ID NO: 2 - DNA, 21 bp, synthetic construct
  misc_feature 1..21: note = Curated E-probe
  source 1..21: mol_type = other DNA
aaattggccggccttacccgg

SEQ ID NO: 3 - DNA, 21 bp, synthetic construct
  misc_feature 1..21: note = Simulated Sample
  source 1..21: mol_type = other DNA
aaattggccggccttacccgg

SEQ ID NO: 4 - DNA, 21 bp, synthetic construct
  misc_feature 1..21: note = Curated E-probe
  source 1..21: mol_type = other DNA
taaatgggcgggcttacccgc